(19) United States
(12) Patent Application Publication
(10) Pub. No.: US 2025/0385542 A1
(43) Pub. Date: Dec. 18, 2025

(54) UNINTERRUPTIBLE RESPIRATORY SYSTEM

(71) Applicant: ZOPEC MEDICAL LLC, Blaine, MN (US)

(72) Inventor: Strong Shihchuang HUANG, Blaine, MN (US)

(73) Assignee: ZOPEC MEDICAL LLC, Blaine, MN (US)

(21) Appl. No.: 19/175,888

(22) Filed: Apr. 10, 2025

Related U.S. Application Data
(60) Provisional application No. 63/659,936, filed on Jun. 14, 2024.

(51) Int. Cl. H02J 9/06; A61M 16/00; H01M 10/48; H01M 10/6572; H02J 7/00

(52) U.S. Cl. CPC H02J 9/062; A61M 16/0051; A61M 16/022; H01M 10/488; H02J 7/0048; H02J 7/0063; H02J 7/00714; A61M 2205/18; A61M 2205/8206; A61M 2205/8262; H01M 10/6572

(57) ABSTRACT

An uninterruptible power supply system with a housing, at least one input port configured to receive power from an external power source, at least one output port configured to provide power to a respiratory device, a battery pack contained within the housing, a power supply circuit configured to selectively bypass the battery pack and enable the power from the external power source to provide power to the respiratory device, while allowing power to charge the battery pack of the power supply; and a warning system configured to provide an indication of the power level of the battery pack. The uninterruptible power supply system allowing locking of cables inserted into the input and output ports. The uninterruptible power supply system allowing daisy-chaining to improve the total power level.

### CROSS-REFERENCE TO RELATED APPLICATION(S)

This application claims priority to and the benefit of U.S. Application No. 63/659,936, filed on Jun. 14, 2024, titled UNINTERRUPTIBLE RESPIRATORY SYSTEM, the disclosure of which is hereby incorporated by reference in its entirety.

### BACKGROUND

Respiratory devices such as nebulizers, high-frequency oscillatory ventilation (HFOV) devices, and nasal high flow therapy (HFNC) devices are used in emergency situations to assist critical-care patients to breathe or inhale medicine. Often these respiratory devices are plugged into an external power source such as wall outlets or power receptacles in emergency transport vehicles. However, wall outlets and emergency transport vehicles may cease functioning. Further, patients connected to these respiratory devices may need to be transported. The respiratory devices may be unable to be moved due to concerns with power supply to the respiratory device. If these machines cease operating, critical-care patients could have difficulty breathing or be left unable to breath entirely.

### SUMMARY

In general terms, this disclosure is directed to an uninterruptible respiratory system. In some embodiments, and by non-limiting example, the uninterruptible respiratory system includes an uninterruptible power supply system that provides uninterruptible power to a respiratory device.

One aspect is an uninterruptible power supply system comprising: a housing; at least one input port configured to receive power from an external power source; at least one output port configured to provide power to a respiratory device; a battery pack contained within the housing; a power supply circuit configured to selectively bypass the battery pack and enable the power from the external power source to provide power to the respiratory device, while allowing power to charge the battery pack of the power supply; and a warning system configured to provide an indication of the power level of the battery pack.

Another aspect is an uninterruptible respiratory system comprising: a respiratory device including a hose and a mask, the respiratory device configured to provide heated pressure through the hose to the mask; and a power supply including: a housing; at least one input port configured to receive power from an external power source; at least one output port configured to provide power to a respiratory device; a battery pack contained within the housing; a power supply circuit configured to selectively bypass the battery pack and enable the power from the external power source to provide power to the respiratory device, while allowing power to charge the battery pack of the power supply; a warning system configured to provide an indication of the power level of the battery pack; and a controller configured to operate the warning system.

A further aspect is an uninterruptible battery pack comprising: a housing; at least one input port configured to receive power from an external power source; at least one output port configured to provide power to a respiratory device; a battery pack configured to receive power from an external power source through the input port; a power supply circuit configured to selectively bypass the battery pack and enable the power from the external power source to provide power to the respiratory device, while allowing power to charge the battery pack of the power supply; a warning system configured to provide an indication of the power level of the battery pack; and a controller configured to operate the warning system, a bypass, and charging of the power within the power supply circuit.

### BRIEF DESCRIPTION OF THE DRAWINGS

FIG. 1 is a perspective view of an example uninterruptible respiratory system.

FIG. 2 is a diagram illustrating an example respiratory device.

FIG. 3 is a diagram illustrating airflow through an example respiratory device.

FIG. 4 is a perspective view of an example respiratory device attached to a stand for transport.

FIG. 5 is a perspective view of an example patient transport using the power supply system.

FIG. 6 is a perspective view of an uninterruptible power supply system.

FIG. 7 is a side view of a front side of the uninterruptible power supply system of FIG. 6.

FIG. 8 is a side view of a back side of the uninterruptible power supply system of FIG. 6.

FIG. 9 is a perspective view of a locking mechanism on the front side of the uninterruptible power supply system of FIG. 6.

FIG. 10 is a side view of a left side of the uninterruptible power supply system of FIG. 6.

FIG. 11 is a side view of a bottom side of the uninterruptible power supply system of FIG. 6.

FIG. 12 is a top view of a bracket for mounting the uninterruptible power supply system of FIG. 6.

FIG. 13 is a side view of a bracket for mounting the uninterruptible power supply system of FIG. 6.

FIG. 14 illustrates a perspective view of a case for the uninterruptible power supply system 104.

FIG. 15 illustrates a side view of the bottom of the case for the uninterruptible power supply system 104.

FIG. 16 is a side view of a groove on a bottom side of the uninterruptible power supply system of FIG. 6.

FIG. 17 shows a cross sectional view of an alternate embodiment of the case for the uninterruptible power supply system.

FIG. 18 is a cross sectional view of an example cooling system of the uninterruptible power supply system of FIG. 6.

FIG. 19 is a diagram illustrating an example uninterruptible power supply.

FIG. 20 is a schematic of an example power supply circuit.

FIG. 21 illustrates an example method of controlling power indicating lights.

FIG. 22 illustrates an example method of controlling a plurality of warning alarms.

### DETAILED DESCRIPTION

Various embodiments will be described in detail with reference to the drawings, wherein like reference numerals represent like parts and assemblies throughout the several views. Reference to various embodiments does not limit the scope of the claims attached hereto. Additionally, any examples set forth in this specification are not intended to be limiting and merely set forth some of the many possible embodiments for the appended claims.

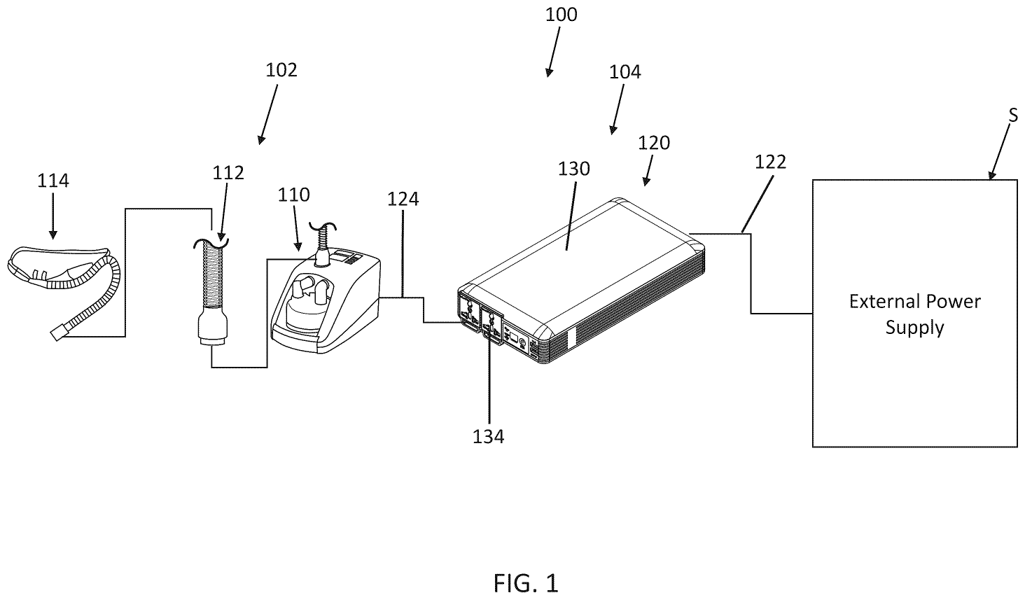

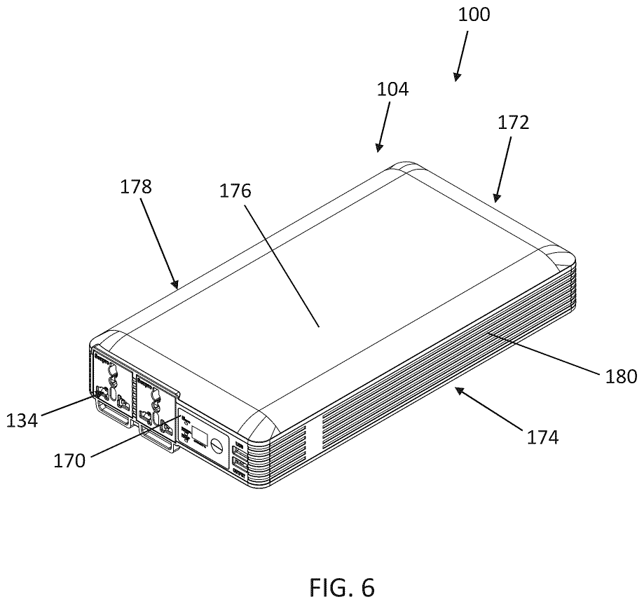

FIG. 1 is a perspective view of an example uninterruptible respiratory system 100. The example uninterruptible respiratory system 100 includes a respiratory device 102 and an uninterruptible power supply system 104. The respiratory device 102 includes a respiratory machine 110, a hose 112, and a patient interface 114. The example uninterruptible power supply system 104 includes a power supply 120, a power cable 122, and an output cable 124. The example power supply 120 includes a housing 130, at least one input port 132 (shown in FIG. 6), and at least one output port 134. An external power source S is also shown in FIG. 1.

In the illustrated example, the example uninterruptible respiratory system 100 includes a respiratory device 102 and an uninterruptible power supply system 104. The respiratory device 102 provides assistance to a patient's respiratory system by assisting in breathing, supply of medication, or humidified air. The respiratory device 102 can be various respiratory devices 102 depending on a patient's condition. If power is lost while the respiratory device 102 is in use, the respiratory device 102 cannot operate to support breathing or deliver medicine depending on the device 102. In order to avoid such disruptions, the uninterruptible respiratory system 100 includes the uninterruptible power supply system 104 that functions to maintain a continuous supply of power to the respiratory device 102 while the device is in use (such as during a power outage or during patient transport), so that the operation of the respiratory device 102 is not interrupted.

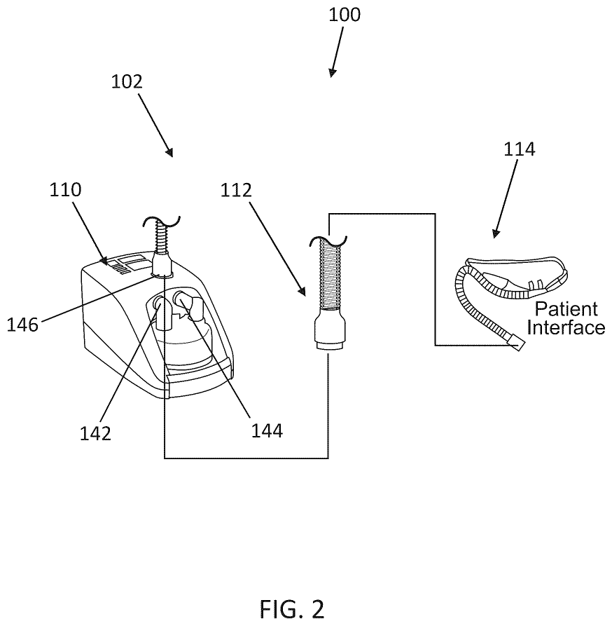

In some embodiments, the respiratory device 102 includes a respiratory machine 110, a hose 112, and a patient interface 114. The respiratory device 102 operates to provide positive pressure air, medication, and/or humidified air to a user. In some embodiments, the respiratory machine 110 generates pressurized air and provides the pressurized air through the hose 112 to the patient interface 114 where it is then delivered to a user. For example, the pressurized air can be provided to the upper respiratory tract of the user, such as through the nose and/or the mouth. Examples of the respiratory device 102 are illustrated and described in further detail herein with reference to FIGS. 2-4.

The uninterruptible power supply system 104 provides uninterrupted power to the respiratory device 102 in order to maintain the continuous operation of the respiratory device 102 even in the absence of an external power source S, or in the event that power is lost from the external power source S. The example uninterruptible power supply system 104 includes a power supply 120, a power cable 122, and an output cable 124. The power supply 120 includes at least one input port 132 (shown in FIG. 6) and at least one output port 134.

The power supply 120 is the portion of the uninterruptible power supply system 104 that receives, stores, and outputs power to the respiratory device 102. The example power supply includes a battery pack, which can be charged with power from the external power source S and can subsequently supply the stored power to the respiratory device 102. In some embodiments, when the power supply 120 is connected to the external power source S, the power supply 120 supplies power to the respiratory device 102, while bypassing the battery pack. In some embodiments, the power supply 120 delivers power from an external power source S and from the battery pack, such as to collectively provide even greater power output, whereas in other embodiments the power supply 120 delivers power selectively from either the external power source S or from the battery pack.

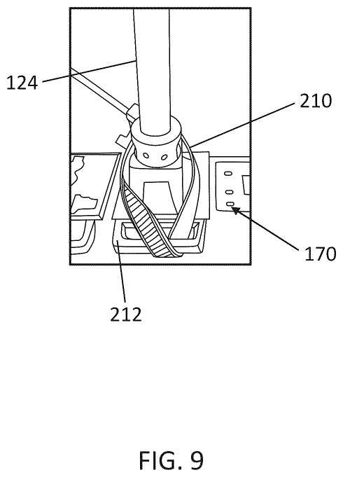

The input port 132 provides a connection for receiving the power cable 122, to receive power from the external power source S. The output port 134 provides a connection for receiving an output cable 124 of the respiratory machine 110, to supply power from the power supply 120 to the respiratory device 102. The power supply 120 is illustrated and described in further detail with reference to FIGS. 6-17.

The power cable 122 receives power from an external power source S for delivery to the power supply 120. The AC power is then supplied to the power supply 120 directly with the power cable 122. In some examples, the external power source S can be one or more additional uninterruptible power supply system 104 which are daisy chained to enlarge the available level of stored energy. Advantageously the total available level of energy is increased and provides a larger length of time for patient transport. The daisy chaining will be described in greater detail later.

The present disclosure refers to example embodiments in which the uninterruptible power supply system 104 provides power to the respiratory device 102. In other embodiments, the uninterruptible power supply system 104 can instead provide power to another electronic device other than the respiratory device 102. An example of another electronic device is a medical device. An example of a medical device is the respiratory device 102. The electronic device can include a device other than a medical device, such as a non-medical device. Accordingly, within the present disclosure, the term respiratory system 100 can, when the context permits, be replaced with the terms electronic system or medical device system to describe other embodiments that are within the scope of the present disclosure. Additionally, the term respiratory device 102, when the context permits, can be replaced with the terms electronic device or medical device to form additional embodiments according to the present disclosure.

FIG. 2 is diagram illustrating a respiratory device 102 connectable to the uninterruptible respiratory system 100. In this example, the respiratory device 102 includes the respiratory machine 110, the hose 112, and the patient interface 114. The respiratory machine 110 includes a power cable port (not shown), an air inlet chamber port 142 and an air outlet chamber port 144, and a hose port 146.

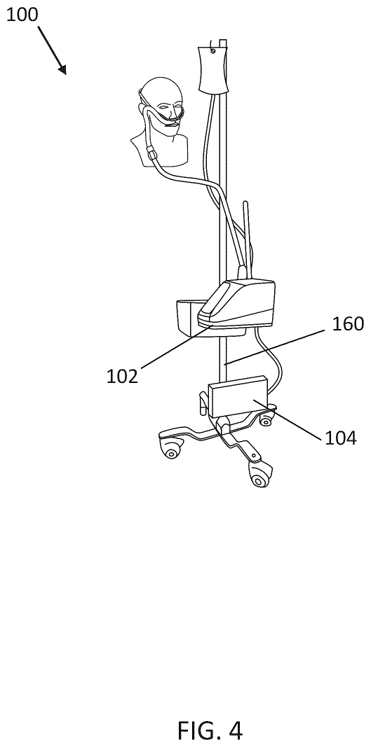

The example respiratory machine 110 is powered by the output cable 124 inserted into power cable output port 134 of the uninterruptible power supply system 104. Air may be pumped through the respiratory machine 110 through the inlet and outlet chamber ports, 142,144 as will be described in greater detail in FIG. 4. The hose 112 is inserted into the hose port 146 at a first end of the hose 112. The second end of the hose 112 is joined to the patient interface 114. The patient interface 114 can be inserted into the nose or over the mouth and nose of a patient. In some instances, the patient interface 114 is a mask. In other examples, the patient interface 114 is a set of nasal tubes. Accordingly, the respiratory machine 110 drives air flow through the hose 112 to the patient interface 114 to provide medication or air to the user.

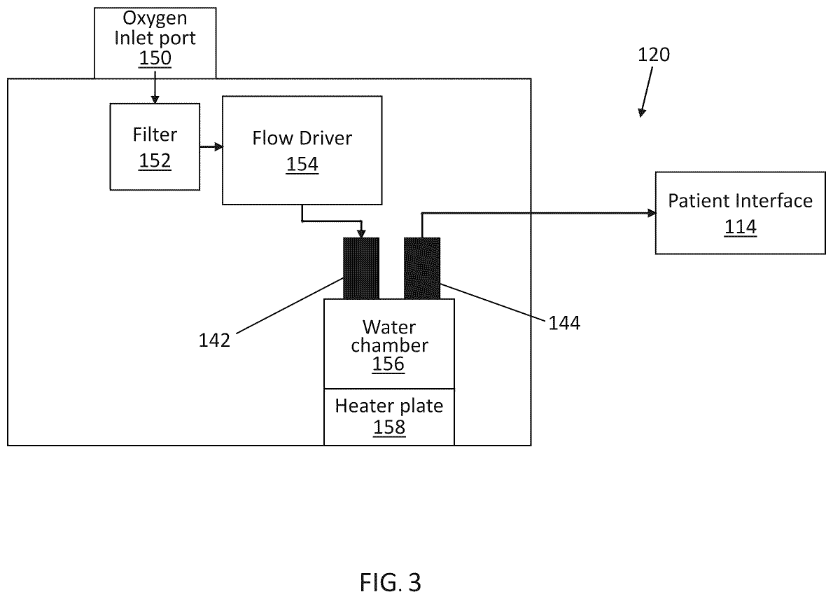

FIG. 3 is a block diagram illustrating the components of the respiratory machine 110. The respiratory machine 110 can be a HFNC machine including an oxygen inlet port 150, a filter, 152, a flow driver 154, a water chamber 156, and a heater plate 158. Air is provided through the oxygen inlet port 150 and through the filter 152 to a flow driver 154. The filter 152 filters air received through the oxygen inlet port 150. In some instances, the flow driver 154 can be a mechanical ventilator. In the example respiratory machine 110, the flow driver 154 is connected to the water chamber 156 at the air inlet chamber port 142. The flow driver 154 generates a pressure to create air flow through the water chamber 156 to deliver air to the patient interface 114. The water chamber 156 is placed on the heater plate 158 and retains water within the respiratory machine 110 to create water vapor for the air. When heated the water chamber 156 may generate humified air with the air flowing through the chamber 156 which is transferred out of the air outlet chamber port 144 to the hose port 146. In the example respiratory machine 110, the hose port 146 is in fluid communication with the water chamber 156 through the port 144. The hose port 146 allows the attachment of the first end of the hose to a housing of the respiratory machine 110.

FIG. 4 is a perspective view of an uninterruptible respiratory system 100 mounted to a stand. The example uninterruptible respiratory system 100 includes a respiratory device 102 and an uninterruptible power supply system 104. Referring to FIG. 4, the uninterruptible respiratory system 100 can be joined to a stand 160. The stand 160 provides a support structure to hold the uninterruptible respiratory system 100. Each of the respiratory device 102 and the uninterruptible power supply system 104 are separately joinable to the stand 160. The stand 160 can be a cart, or other structure capable of holding the respiratory machine 110. An example of the stand 160 is an IV bag stand. In some examples, the stand 160 may be a gurney. In other examples, the uninterruptible respiratory system 100 may be mounted to an emergency transport vehicle. The power supply system 104 may also be joined to the stand 160. As such, the stand 160 may be moved with the patient while holding respiratory device 102 and the power supply system 104. The power supply system 104 allows the respiratory device 102 to remain powered during loss of power or while transporting a patient where a connection to an external power source cannot be maintained.

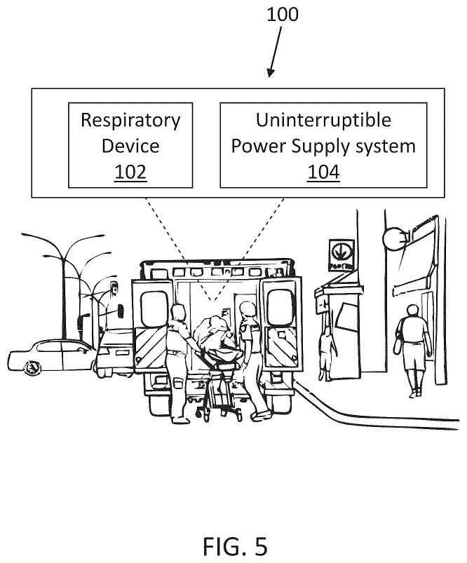

FIG. 5 is a perspective view of an uninterruptible respiratory system 100 used in the transport to an emergency transport vehicle. For instance, as shown in FIG. 5 patients may be in a critical condition but need to be transported from one hospital to another. The patient is transported to an emergency transport vehicle where the power supply system 104 can reconnect to a power source of the emergency transport vehicle. It is understood that other scenarios of patient transport can arise. Another non-limiting example, a transport scenario can include medical personnel responding to a medical emergency in which the patient needs to be connected to a respiratory device 102. The power supply system 104 allows the respiratory device 102 to operate until reaching an emergency transport vehicle. The example emergency transport vehicle is an ambulance; however, the vehicle can be any type of vehicle, such as a helicopter. Importantly, the example power supply system 104 receives signals as a pure sine wave, which can be accepted from emergency transport vehicles without damaging the vehicle's power source. Examples of the power supply system 104 providing a pure sine wave will be described in greater detail later. Additionally, the uninterruptible power supply system 104 has a weight of no greater than 5 lbs. Advantageously, the uninterruptible power supply system 104 allows a lightweight system capable of being easily transported in emergencies.

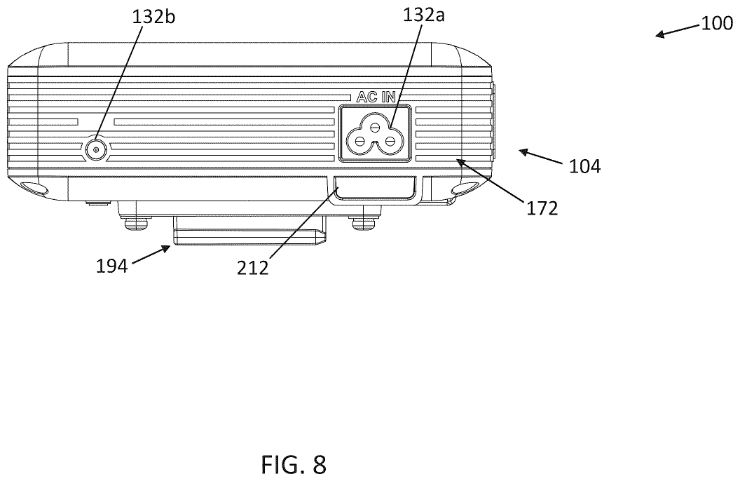

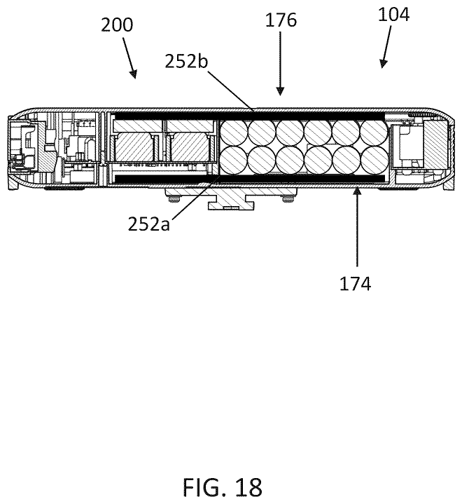

FIGS. 6-9 illustrates an example perspective view of the uninterruptible power supply system 104. The power supply 120 includes the housing 130. The housing 130 retains the battery pack. The housing 130 is flame retardant and explosion proof. The housing 130 can include an AC input port 132a, a DC input port 132b, two AC output ports 134a, b, a display 190, USB ports 192, a locking mechanism 194, power indicating lights 196, a mounting adapter 198 (shown in FIG. 8), and a cooling system 200 (shown in FIG. 18). In some examples. The housing 130 may also include a handle. Referring to FIG. 6, the housing 130 may include a front side 170, a back side 172, a bottom side 174, a top side 176, a left side 178, and a right side 180. The sides define the exterior of the housing 130 and facilitate the positioning of the housing 130 and cables 122, 124 for the power supply 120. The front side 170 may be opposite the back side 172, the bottom side 174 may be opposite the top side 176, the left side 178 may be opposite the right side 180.

The example display 190 provides an indication of the power level of the battery. The locking mechanism 194 is configured to prevent undesired removal of power cables from the power supply. The example mounting adapter 198 allows the power supply system 104 to be mounted on different objects, such as the stand 160. The power indicating lights provide an additional indication of power level within the power supply. The USB ports 192 are configured to output power through conventional USB cables.

Further, the power supply 120 is a medical grade power supply weighing approximately 5 lbs. Medical grade means compliant with standards IEC-60601-1, 62133-2, and 60133-2. As such, the power supply 120 does not emit electromagnetic fields which substantially interfere with other medical devices and the power supply 120 is fireproof. Further, conventional medical grade power supplies weigh 40-50 lbs. Because of the configuration of components used, the power supply 120 has an improved weight which allows for improved transport of patients using respiratory devices 102.

FIGS. 6, 8, and 9 illustrate example front and back sides of the power supply system 104. The front side and back sides may include various input and output ports. In the example front side of FIG. 6, the housing 130 includes an ac input port 132a and an DC input port 132b. In some examples, the DC input port 132b allows an external power source S powered from solar energy to be connected to the uninterruptible power supply system 104. In other examples, the external power source S may be a car charger joined to the DC input port 132b. In the example back side of FIG. 8, the back side includes two AC output ports 134a, b, the USB ports 192, the locking mechanism 194. the display 190 and the power indicating lights 196. In the example uninterruptible power supply system 104, the output ports 134 are universal output ports able to accept multiple types of plugs. The example display 190 is configured to display a numerical value as a percentage of power remaining in the power supply. The housing 130 also includes the power indicating lights 196. In some instances, the power indicating lights are adjacent the display; however, other the lights may be positioned anywhere on the housing. In the example power supply, the power indicating lights 196 are LED lights which can illuminate as green, yellow, or red based on the level of the power remaining. As will be described in greater detail later, a controller within the power supply may monitor the power level of the power supply. The controller 260 may control the illumination of the LED lights to provide an indication to a user of the current power remaining. For instance, green may indicate a first power level threshold between a power level of 95-20%. A yellow light may indicate a second power level threshold of 20%-10%, while the red indicates a third power level threshold below 10%.

As described previously, external power source S can be one or more additional uninterruptible power supply systems 104 which are daisy chained to enlarge the available level of stored energy. When daisy chaining the uninterruptible power supply systems 104 together, the first uninterruptible power supply system 104 in the chain supplies power through bypasses of the uninterruptible power supply system 104 to the respiratory device 102. The controller 260 of each power supply system 104 may determine if the power is not supplied from the power supply system 104 before each respective power supply system 104 in the chain. When a power supply system 104 detects a lack of received power, the power supply system 104 begins supplying power as the external power source S, while the remaining power supply systems 104 downstream continue to bypass power to the respiratory device 102.

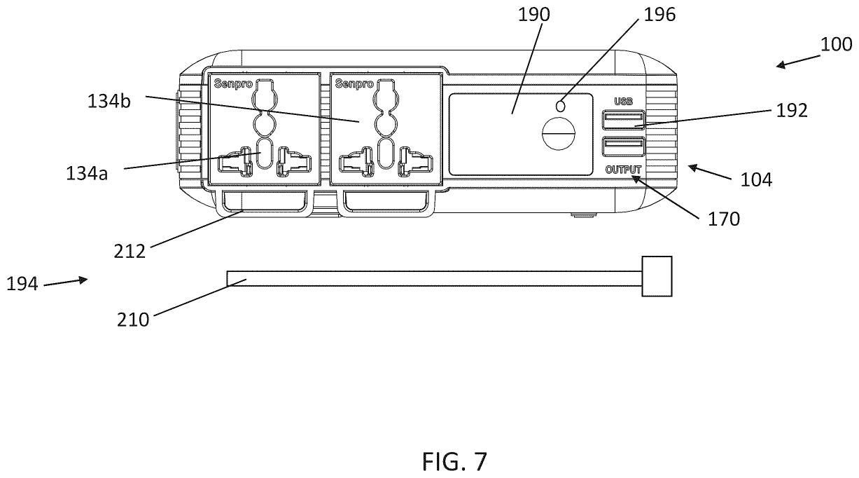

The example locking mechanism 194 is adjacent the AC output ports 134a, b on the back side. While shown on only the back side, it is understood the locking mechanism 194 may be placed adjacent any input port output port, or USB port on the housing. The locking mechanism 194 may include a locking member 210 and a locking structure 212. As shown in FIG. 7, the example locking mechanism 194 is configured to allow a locking member 210 to hold the respiratory device's power cable 124 within either one of AC output ports 134a, b and prevent removal while the locking member 210 is engaged. In the example locking mechanism 194, the locking structure 212 is a locking loop integrally formed as part of the housing. The locking loop may protrude from the back side 172 and be transverse to the bottom side 174. In some examples, the locking structure 212 may be immediately adjacent the output ports 134a, b. In other examples the locking structure 212 may be offset from the output ports 134a, 134b at the bottom side 174. The locking member 210 may be a reusable zip tie; however, other ties or locking configurations may be used. The locking member 210, such as the zip tie, may be inserted through the locking structure 212 (i.e., the locking loop) and around a cable to hold the cable within a respective port. The zip tie may also be provided a color different from the cable and/or housing to allow the locking mechanism to be clearly visible.

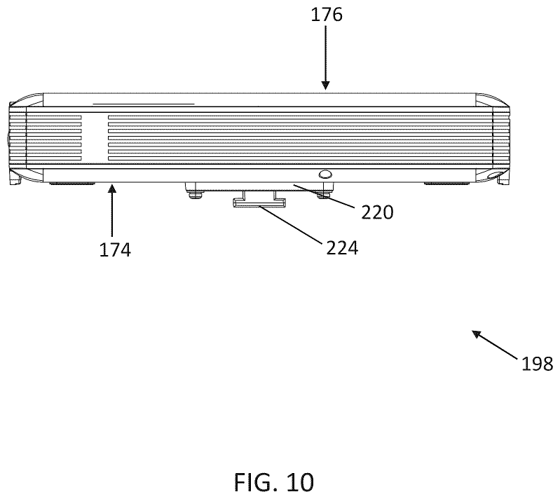

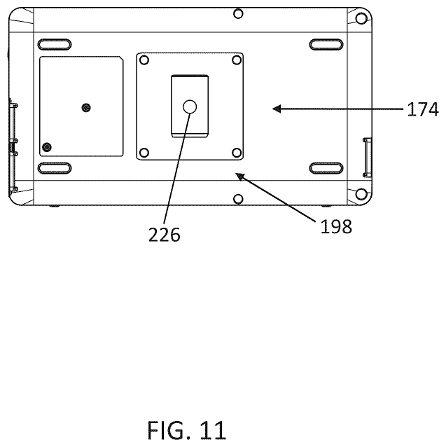

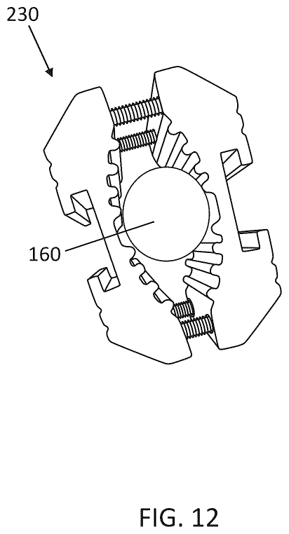

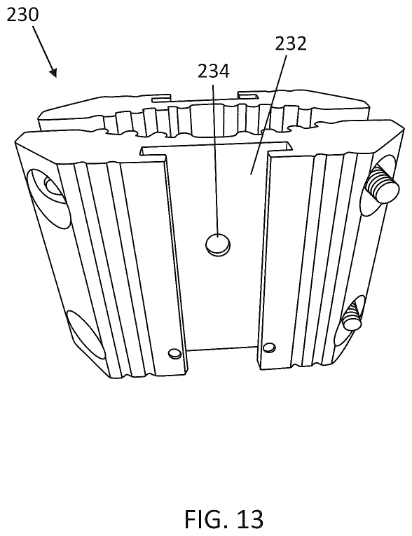

FIGS. 10-11 illustrates views of an example mounting adapter 198 on the bottom side of the power supply. FIG. 11 is a side view of a bottom side of the uninterruptible power supply system of FIG. 6. The example mounting adapter 198 includes a back plate 220, and a mount protrusion 224. The mounting adapter 198 is configured to be received by a mounting bracket 230, which will be described in greater detail in FIGS. 12 and 13. The back plate 220 can be fastened to the bottom side of the housing to provide the mount at either a first or second orientation. As an example, the first orientation allows the length of the bottom side to be mounted vertically on a stand. An example of the second orientation allows the length of the bottom side to be mounted horizontally on the stand 160. The mounting adapter 198 is configured to hold the uninterruptible power supply system on the stand 160. The mounting protrusion 224 is further configured to withstand forces or weight. If an excessive force or weight is applied to the mount, the adapter is configured to break before the housing. This breakaway of the adapter prevents damage to the housing and allows easy replacement of the mount protrusion 224 and back plate 220. The adapter 198 may include a hole 226 for set screws to lock the adapter 198 in place.

FIG. 12 illustrates a top view of the mounting bracket 230 positioned on a stand 160. FIG. 13 illustrates a side view of the mounting bracket 230. Referring to FIG. 12, two mounting brackets 230 may be joined together around the stand 160 in a clamped configuration. In some examples, a single mounting bracket 230 may be attached to the stand 160.

Referring to FIG. 13, the mounting bracket include a mounting portion 232 and a hole 234. The mounting bracket 230 receives the mounting adapter to hold the uninterruptible power supply system 104 on a stand 160. The mounting adapter 198 may fit within the mounting portion 232 of a corresponding mounting bracket 230 attached to a pole of stand 160 or other structure. The hole 234 allows the set screw to be inserted through the mounting bracket 230 and into the mounting adapter to lock the mounting adapter 198 to the bracket 230. The mounting protrusion 224 may be similarly shaped to the mounting portion 232 on the mounting bracket 230. The mounting bracket 230 may be joined to any surface of the stand 160. In the example stand 160, the mounting bracket 230 is attached to a pole of the stand 160. The uninterruptible power supply system 104 is able to then be positioned on the stand 160 to allow transport of the patient while power supply system 104 powers the respiratory device 102.

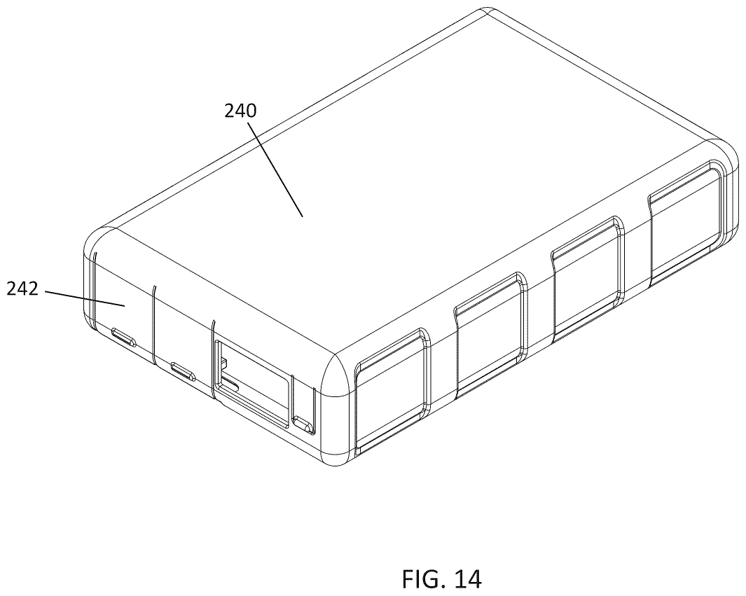

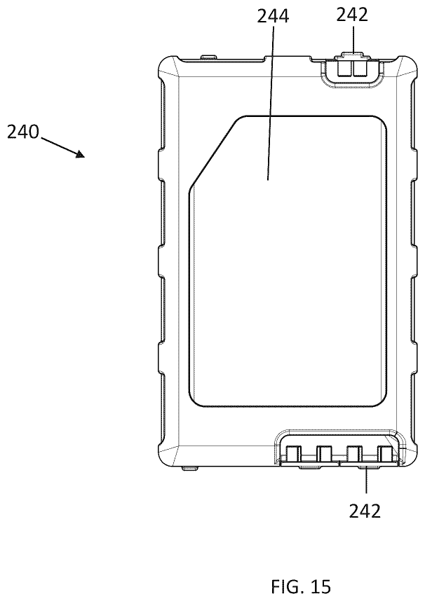

FIG. 14 illustrates a perspective view of a case for the uninterruptible power supply system 104. FIG. 15 illustrates a bottom view of the case. The case 240 includes flaps 242 and a cutout 244 (shown best in FIG. 15). The case 240 is made of silicon and provides a protective layer to the housing 130. Additionally, the case 240 allows the uninterruptible respiratory system 100 to be mounted while still providing protection. The flaps 242 cover the at least one input port 132 and the at least one output port 134. Further, the flaps 242 include protrusions 246 on the interior of the case 240, which are shaped to extend into the respective input and output ports 132, 134. When the protrusion is received within the respective input and output ports 132, 134, the flaps provide waterproofing to the power supply system 104. The cutout allows the case 240 to be flexed around the exterior of the housing 130 to cover the housing. The cutout is positioned around the mounting adapter 198 when joined to the housing 130 and allows the mounting adapter to engage the mounting bracket 230.

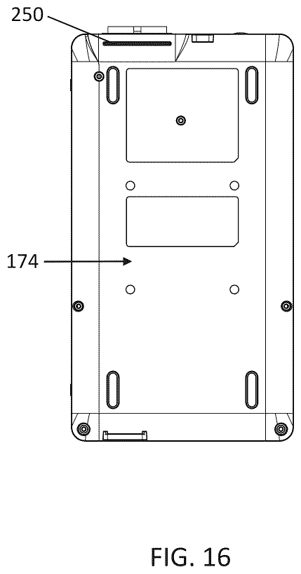

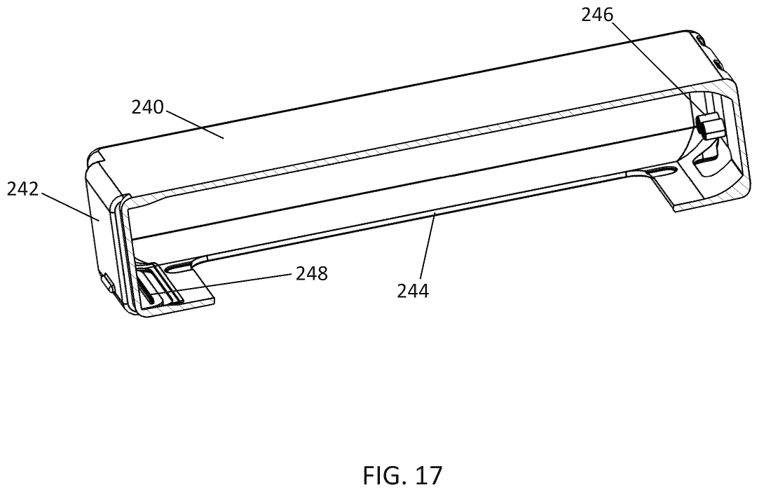

FIG. 16 illustrates a bottom view of the uninterruptible power supply system 104. In the example uninterruptible power supply system 104, the bottom side 174 can include a groove adjacent the output ports 134a, b. As will be described in further detail in FIG. 17, the groove 250 may be used in engaging a case which protects the housing 130. As a non-limiting example, flaps of the case may engage the groove to close the output ports 134a, b.

FIG. 17 shows a cross sectional view of an alternate embodiment of the case 240 for the uninterruptible power supply system 104. The case 240 includes flaps 242 and a cutout 244. The flaps 242 cover the input and output ports 132, 134. Further, the flaps 242 further include a lip 248 at a distal end which grip the groove 250 on the housing 130. When the lip 248 is engaged with the groove 250 (shown in FIG. 12), the flap 242 is retained in a closed position and provides waterproofing to the power supply 120.

FIG. 18 illustrates a cross sectional view showing the cooling system 200. The power supply system 104 also includes the cooling system 200 within an interior of the housing 130. The cooling system configured to use solid state Peltier cooling system. The solid state Peltier cooling system is configured as two Peltier coolers 252a, b. A first Peltier cooler 252a is positioned between a front side 170 of the housing 130 and a battery pack 268, and a first Peltier cooler 252b positioned between a back side of the housing and the battery pack. The first side of the battery preferably is adjacent the bottom side of the housing. The second side of the battery pack is preferably opposite the first side of the battery pack and adjacent the top side of the housing. Beneficially, heat is exchanged from the battery pack to the housing and is dissipated through the coolers 252a, b. A further benefit is that no fan is required, and sterilization of the interior is not required in medical applications. For instance, hospitals often require the cleaning of medical devices after use in treating a patient. Without a fan, the housing 130 does not include any openings to the interior of the power supply 120 and does not require internal sterilization of the power supply between uses.

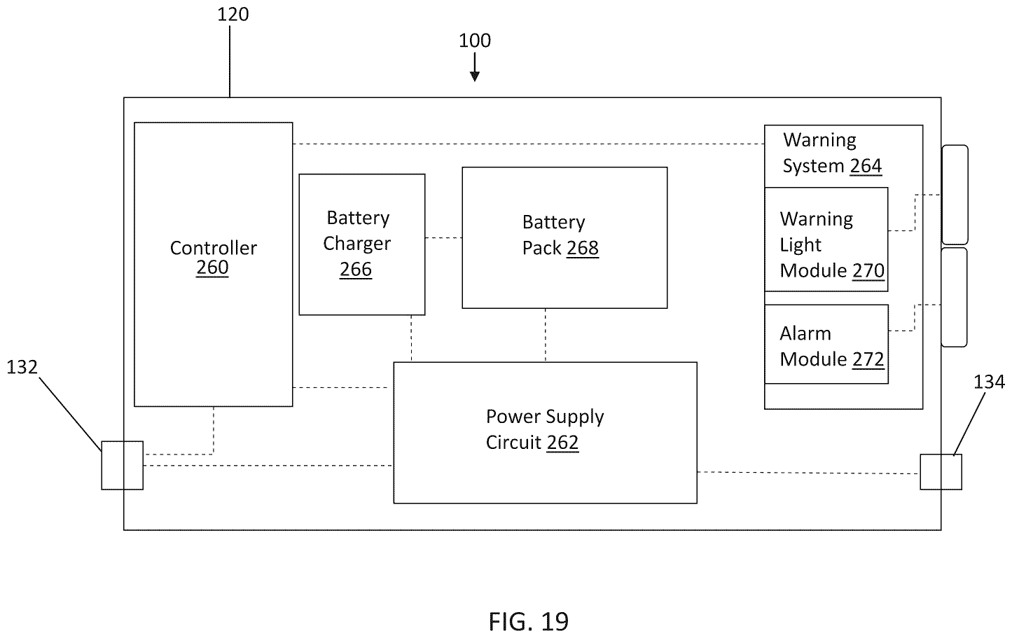

FIG. 19 illustrates an example block diagram of the uninterruptible power supply system 104 of the uninterruptible respiratory system 100. The example uninterruptible power supply system 104 includes a power supply 120, a controller 260, a power supply circuit 262, and a warning system 264. The power supply circuit 262 can include a battery charger 266, a battery pack 268. The power supply circuit 262 allows the uninterruptible power supply 120 to bypass power directly to the respiratory device 102 or allows the charging of the battery pack 268. The battery charger 266 converts energy received from an external power source S to charge the battery pack 268. The input port 132 charges the uninterruptible power supply system 104 at 45-watt hours at a total 300-watt hour charge. The battery pack 268 provides a source of power for the respiratory device 102 incase power to the external power source S is interrupted or lost.

In the example system 100, the power supply circuit 262 is in communication with the input port 132 and the output ports 134a, b. The power supply circuit 262 is in further communication with the battery charger 266. The battery charger 266 is connected to the battery pack 268 to receive and convert energy from the power supply circuit 262 to the battery pack 268. The warning system 264 is in communication with the controller 260, a warning light module 270, and an alarm module 272. The example warning light module 270 controls the operation of the power indicator lights. The alarm module 272 is configured to control a plurality of alarms.

The warning light module 270 allows different lights to be illuminated to provide indications of power level of the battery. As described previously some examples may include LED lights which can be illuminated in different colors. In some instances, the different colors may indicate different threshold of power remaining within the battery pack.

In the example power supply 120, the power indicating lights 196 are LED lights which can illuminate as green, yellow, or red based on the level of the power remaining. The controller 260 within the power supply 120 may monitor the power level of the power supply. The controller 260 may control the illumination of the LED lights to provide an indication to a user of the current power remaining. The controller 260 may send a signal to the warning light modules to switch the color of the LED lights. As a non-limiting example, the lights may illuminate green, yellow, or red. For instance, green may indicate a first power level threshold between a power level of 95-20%. A yellow light may indicate a second power level threshold of 20%-10%, while the red indicates a third power level threshold below 10%.

The alarm module 272 controls the plurality of alarms to warn users of low power levels of the uninterruptible power supply system 104 which if no charged may lead to operation of the respiratory device 102 being interrupted. It should be understood that speakers may provide the alarms. Each alarm provides a different audible noise and indicating a different level of level of stored power. The audible noise is a continuous noise and may be mutable. In some instances, the audible noise may a different tone or quicker repetition of the audible noise. The warning system is configured to begin one of the alarms when a predetermined level of stored power is reached.

In some examples, a first alarm indicates a first predetermined level of power remaining, while a second alarm indicates a second predetermined level of power remaining lower than the first predetermined level. In some examples, a button may be pressed by a user to silence the first or second alarm. Preferably, both the first and second alarms are continuous until silenced by a user. In some examples, the second alarm may be quicker repeating tone and/or louder than the first alarm. In other instances, the second alarm is a different tone.

The warning system further includes a lower limit alarm. When a lower limit of power remaining is reached, the lower limit alarm is triggered, and the lower limit alarm continues until the uninterruptible respiratory system 100 receives power from the external power source and the controller prevent muting of the lower limit alarm by pressing the button. The lower limit alarm may also have a different tone, faster tone, and/or louder tone than the first and/or second alarm. In some examples, the alarm may be a repeatable recording. For instance, the recording may state to reconnect the battery or that the battery is critically low. The lower limit of power remaining may be 5% or below.

Because the lower limit alarm is unable to muted unless connection to an external power source is restored, the lower limit alarm reduces the likelihood the battery pack runs out of power before being plugged into an externa power source. The lower limit alarm is particularly advantageous in medical transport scenarios where a patient may often be relying on life-sustaining medical devices which require power to be maintained without interruption. Further the uninterruptible power supply 104 may switch to supply power within 12 ms of loss of power to an external power source. The example lower limit alarm provides a final alarm which cannot be silenced as a final warning, while providing multiple prior warnings of battery level.

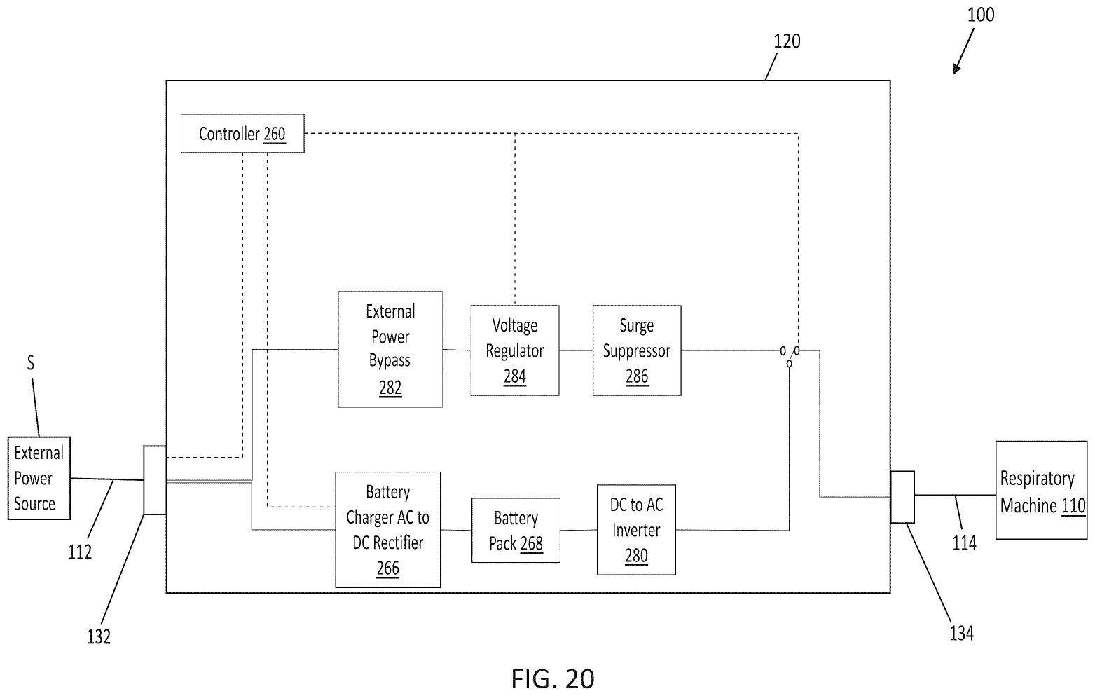

FIG. 20 illustrates an example block diagram of the power supply circuit. The power supply circuit 262 includes the battery charger 266, a battery pack 268, a DC to AC invertor 280, an external power bypass 282, a voltage regulator 284, a surge suppressor 286, and a switch 288. In some instances, the external power bypass may be referred to as a passthrough.

The external power bypass 282 allows external power supplied at the input port 132 to bypass the battery and be used to directly power the respiratory device 102. The voltage regulator 284 regulates the voltage at a fixed voltage. The surge suppressor 286 suppresses surges or spikes of voltage in the external power bypass 282. The controller 260 is in communication with the power supply circuit 262, input port 132, battery charger 266, battery pack 268 and a switch 288. The controller 260 is able to switch been supplying power received at the input port 132 to the external power bypass 282 or to the battery charger 266 When switched to supply power to the battery charger 266, the battery charger 266 recharges the battery pack 268. Additionally, the controller 260 is in communication with the switch 288 to switch between whether the power is supplied from the bypass 282 or battery pack 268 to the at least one output port 134. As such, the uninterruptible power supply system 104 of the uninterruptible respiratory system 100 operates as a line interactive uninterruptible power supply. The power supply 120 provides back-up power and utilizes battery power to power the respiratory device 102 when either no external power source is connected or a power outage is detected. When an external power source S is supplying power to the uninterruptible power supply system 104, the external power bypass 282 is used to supply power to the respiratory device 102.

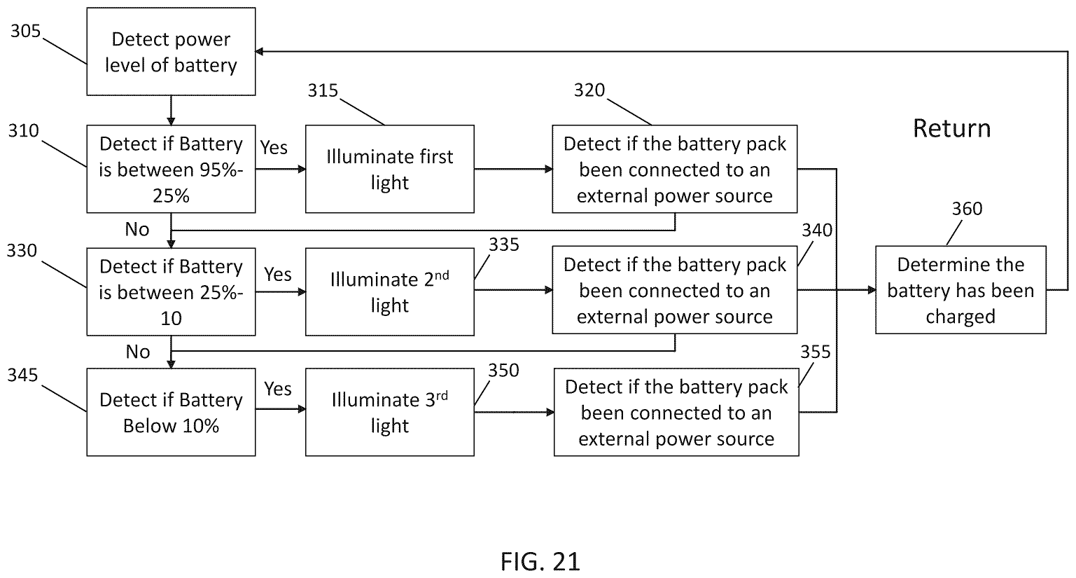

FIG. 21 is a flow diagram of a method of controlling the power indicator lights. The method 300 includes operations 305, 310, 315, 320, 330, 335, 340, 345, 350, 355, and 360. The operation 305 is performed to detect the power level of the battery. For instance, the controller 260 may detect the power level by coulomb counting.

The operation 310 detects if the battery level is between a first range. An example range may be 95% to 25%. It is understood the first range may vary in other examples. If no, the method proceeds to operation 330. If yes, operation 315 illuminates the first light. The method proceeds to operation 320 which detects if the battery pack has been connected to an external power source. If the battery back has been connected to an external power source, the method proceeds to operation 360 which determines the battery pack has been recharged and returns to operation 305.

The operation 330 detects if the battery level is lower than a second range. An example range may be a range below 25% and higher than 10%. It is understood the second range may vary in other examples. If no, the method proceeds to operation 340. If yes, operation 330 illuminates the second light. The method then proceeds to operation 335 detects if the battery back has been connected to an external power source. If the battery back has been connected to an external power source, the method proceeds to operation 360 which determines the battery pack has been recharged and returns to operation 305. If no, the method proceeds to operation 340.

The operation 345 detects if the battery level is lower than a third range. An example range may be 10% or below. It is understood the third range may vary in other examples. If no, the method proceeds to operation 355. If yes, operations 345 illuminates the third light. If the battery back has been connected to an external power source, the method proceeds to operation 360 which determines the battery pack has been recharged and returns to operation 305.

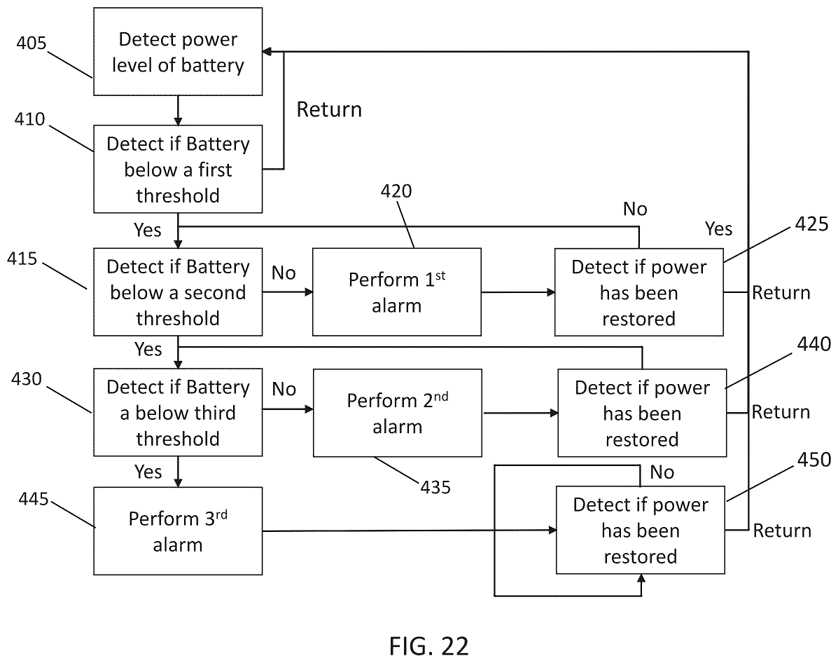

FIG. 22 is a flow diagram of a method of controlling the warning alarms. The method 400 includes operations 405, 410, 415, 420, 425, 430, 435, 440, 445, and 450. The operation 405 to detect the power level of the battery pack. For instance, the controller 260 may determine the detect the power level by coulomb counting. The operation 410 includes detecting if the battery pack power level is below a first threshold. In the example method 400, the first threshold may be 20%. It is understood the first threshold value may vary in other examples. If not below the 20%, the controller returns to operation 405. If below the first threshold, operation 415 detects if the battery is below a second threshold. In the example method 400, the second threshold may be 10%. If not below the second threshold, operation 420 sounds a first alarm. The first alarm indicating a power level below 20%. In the example method, the first alarm may be muted by a user. The operation 425 then determines if power has been restored. If not, the operations 415, 420, 425 loop until the battery pack is detected to fall below the second threshold or power is restored. If an external power source has been connected, the method returns to operation 405.

If the power level falls below the second threshold, operation 430 detects if the battery is below a third threshold. In the example method 400, the second threshold may be 10%. It is understood the second threshold value may vary in other examples. If not below the third threshold, operation 435 sounds a second alarm. The second alarm indicating a power level below 10%. In the example method, the second alarm may be muted by a user. The operation 440 then determines if power has been restored. If not, the operations 430, 435, 440 until the battery pack is detected to fall below the third threshold or power is restored. If an external power source has been connected, the method returns to operation 405.

If the power level falls below the third threshold, operation 430 detects if the battery is below a third threshold. In the example method 400, the third threshold may be 5%. It is understood the third threshold value may vary in other examples. If not below the second threshold, operation 445 sounds a third alarm. The third alarm indicating a power level below 5%. In the example method, the third alarm may not be muted by a user. The third alarm is configured to continue until an external power source has been connected. The operation 440 then determines if power has been restored. If not, the operation 450 is configured to loop until an external power is connected. If an external power source has been connected, the method returns to operation 405 and the alarm is stopped.

What is claimed is:

1. An uninterruptible power supply system comprising:
a housing;
at least one input port configured to receive power from an external power source;
at least one output port configured to provide power to a respiratory device;
a battery pack contained within the housing;
a power supply circuit configured to selectively bypass the battery pack and enable the power from the external power source to provide power to the respiratory device, while allowing power to charge the battery pack of the power supply; and
a warning system configured to provide an indication of the power level of the battery pack.

2. The uninterruptible power supply system of claim 1, wherein the power supply system has a weight of no greater than 5 lbs. and receives an AC power of 110 v or 230V.

3. The uninterruptible power supply system of claim 1, wherein a circuit of the power supply includes a current sensor configured to detect when power is not being received through the AC power input and supplying AC power from the power supply to a connectable external device.

4. The uninterruptible power supply system of claim 1, further including a warning system for indicating a level of stored energy of the power supply, the warning system including a plurality of lights, the plurality of lights indicating the level of stored energy of the power supply and each light indicating different levels of stored energy.

5. The uninterruptible power supply system of claim 1, the warning system including a plurality of alarms, each alarm being a different audible noise and indicating a different level of level of stored energy, the warning system configured to begin one of the alarms when a predetermined level of stored energy is reached.

6. The uninterruptible power supply system of claim 5, wherein the plurality of alarms includes a first alarm and a second alarm, the first alarm indicates a first level of power remaining, the second alarm indicates a second level of power remaining, and the first and second alarms continue until silenced.

7. The uninterruptible power supply system of claim 5, wherein the plurality of alarms includes a lower limit alarm, and wherein when a lower limit of stored energy is reached, the lower limit alarm is generated, and the lower limit alarm continues until the portable power supply system receives power from an exterior power source.

8. The uninterruptible power supply system of claim 1, wherein the housing is flame-retardant.

9. The uninterruptible power supply system of claim 1, further comprising a mounting adapter attached to the housing, and the mount adapter is joinable with a bracket on a stand for joining the power supply to the stand.

10. The uninterruptible power supply system of claim 1, further comprising a cooling system configured to use solid state Peltier cooling.

11. The uninterruptible power supply system of claim 10, wherein the cooling system includes two Peltier coolers, a first Peltier cooler positioned between a top side of the housing and the battery pack, and a second Peltier cooler positioned between a bottom side of the housing and the battery pack.

12. The uninterruptible power supply system of claim 1, further including a display positioned on the housing and the display is configured to display the remaining percentage of power.

13. The uninterruptible power supply system of claim 1, wherein the housing includes locking loops capable of allowing locking members to be inserted through the locking loops and around a cable connectable within the at least one of the input port or the output port to hold the cable within the input port or output port.

14. The uninterruptible power supply system of claim 13, wherein the locking loops are positioned adjacent at least one of the input port or the output port.

15. The uninterruptible power supply system of claim 1, further comprising a cover for protecting the housing of the power supply.

16. The uninterruptible power supply system of claim 1, wherein the power supply circuit is configured to bypass power from the input port to the respiratory device without recharging the power supply when the portable power supply is fully charged.

17. The uninterruptible power supply system of claim 1, wherein the least one input port includes a first AC input port and a DC input port.

18. The uninterruptible power supply system of claim 1, wherein the output port includes a first AC output port and a second output port.

19. The uninterruptible power supply system of claim 9, wherein the mounting adapter includes a back plate and a mounting protrusion, and wherein the mounting protrusion is configured to break before the housing when a force is applied to uninterruptible power supply system.

20. The uninterruptible power supply system of claim 1, wherein the power supply system does not include a fan.

21. The uninterruptible power supply system of claim 17, wherein the DC input port receives solar power from the external power source or the external power is a car charger.

22. The uninterruptible power supply system of claim 1, wherein the uninterruptible power supply system is configured to be daisy chained to increase total available power during transport.

23. An uninterruptible respiratory system comprising:
a respiratory device including a hose and a mask, the respiratory device configured to provide heated pressure through the hose to the mask; and
a power supply including:
a housing;
at least one input port configured to receive power from an external power source;
at least one output port configured to provide power to a respiratory device;
a battery pack contained within the housing;
a power supply circuit configured to selectively bypass the battery pack and enable the power from the external power source to provide power to the respiratory device, while allowing power to charge the battery pack of the power supply;
a warning system configured to provide an indication of the power level of the battery pack; and
a controller configured to operate the warning system.

24. An uninterruptible battery pack comprising:
a housing
at least one input port configured to receive power from an external power source;
at least one output port configured to provide power to a respiratory device;
a battery pack configured to receive power from an external power source through the input port;
a power supply circuit configured to selectively bypass the battery pack and enable the power from the external power source to provide power to the respiratory device, while allowing power to charge the battery pack of the power supply;
a warning system configured to provide an indication of the power level of the battery pack; and
a controller configured to operate the warning system, a bypass, and charging of the power within the power supply circuit.
